(19) United States
(12) Patent Application Publication
(10) Pub. No.: US 2024/0122555 A1
(43) Pub. Date: Apr. 18, 2024

(54) TROUBLESHOOTING MEDICAL IMAGING EQUIPMENT

(71) Applicant: Siemens Medical Solutions USA, Inc., Malvern, PA (US)

(72) Inventors: John Pawlak, Villa Park, IL (US); Antje Spade, Des Plaines, IL (US); Daniel Dobrescu, Algonquin, IL (US); Bernardo Hermont, Monmouth Junction, NJ (US); Kausar Khan, Bartlett, IL (US); Robert Peter Hurley, III, Knoxville, TN (US); Micah Sovereign, Streamwood, IL (US)

(21) Appl. No.: 18/047,306

(22) Filed: Oct. 18, 2022

(51) Int. Cl. A61B 6/03; G06T 7/00

(52) U.S. Cl. CPC A61B 6/03; G06T 7/0014; G06T 11/003

(57) ABSTRACT

A framework for troubleshooting medical imaging equipment. Log event data and state data of one or more components of the medical imaging equipment are received. One or more calculations are performed based on the log event data and state data to generate processed data. One or more representations of the one or more components of the medical imaging equipment may then be generated based on the processed data, the log event data, the state data, or a combination thereof.

### TECHNICAL FIELD

The present disclosure generally relates to medical imaging equipment, and more particularly to a framework for troubleshooting medical imaging equipment.

### BACKGROUND

The field of medical imaging has seen significant advances since the time X-rays were first used to determine anatomical abnormalities. Medical imaging equipment has progressed from modern machines, such as Magnetic Resonance (MR) imaging scanners, Computed Tomographic (CT) scanners and Positron Emission Tomographic (PET) scanners, to multimodality imaging systems such as PET-CT and PET-MR imaging systems.

Medical imaging equipment may output a tremendous number of log events in a very short period of time (e.g., few minutes). It takes a deep knowledge and a lot of training and experience to determine which log events to ignore or focus on during troubleshooting. It is difficult to determine if a log event is still relevant at the present time. Additionally, it proves difficult to determine if log events are major or causal log events based on other primary issues. Troubleshooting medical imaging equipment with limited capability for digging through thousands of log events to identify a small set of relevant log events is a very difficult task.

### SUMMARY

Described herein is a framework for troubleshooting medical imaging equipment. In accordance with one aspect, log event data and state data of one or more components of the medical imaging equipment are received. One or more calculations are performed based on the log event data and state data to generate processed data. One or more representations of the one or more components of the medical imaging equipment may then be generated based on the processed data, the log event data, the state data, or a combination thereof.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary system;

FIG. 2 shows an exemplary cloud platform for remotely deploying the troubleshooting tool;

FIG. 3 shows an exemplary method of troubleshooting medical imaging equipment;

FIG. 4A shows an exemplary user-interface (UI) screen generated by pattern creator to manage patterns;

FIG. 4B shows another exemplary UI screen generated by pattern creator to define a pattern;

FIG. 5A is a block diagram showing an exemplary user interface screen generated by the troubleshooting tool;

FIG. 5B shows an exemplary representation generated by troubleshooting tool;

FIG. 6A shows an exemplary information panel;

FIG. 6B shows an exemplary diagnostic panel generated by the diagnostic tool;

FIG. 6C shows another exemplary diagnostic panel generated by the diagnostic tool;

FIG. 7 shows an exemplary log events table; and

FIG. 8 shows an exemplary timeline.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed; on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present framework are not described with reference to any particular programming language. It will be appreciated that a variety of programming languages may be used.

Medical imaging equipment log events are the primary means used to troubleshoot imaging systems. Log events retrieved from the system are analyzed, determined if relevant and conclusions are drawn for corrective system action. Log events may also lead one to use system diagnostics to further analyze system health and further troubleshoot. Together along with service personnel training and experience, troubleshooting is achieved.

The present framework provides a tool for troubleshooting medical imaging equipment. In accordance with one aspect, the troubleshooting tool performs off-system pattern analysis of log events and state data and generates a graphical system representation that highlights areas to focus on. The advantage of analyzing patterns as opposed to individual log entries or state values is the ability to identify the root cause of error. In accordance with another aspect, the troubleshooting tool provides an interactive timeline that immediately highlights the primary log events of interest to focus on. Additionally, the troubleshooting tool provides live status data streaming that enables a remote user to see the present system conditions for accurate troubleshooting. If the need arises, on-site service personnel may be dispatched with the correct action plan and/or component(s) needed to fix the medical imaging equipment the first time.

The use of images helps build confidence in service personnel in locating replaceable components. The tool provides dynamic and instant sensor feedback that can expediently facilitate troubleshooting and determining whether sensors are functioning and/or reporting as expected. Software-driven pattern analysis facilitates troubleshooting that allows for repeated and consistent action plans to be followed. Patterns also reside on a server that can be updated based on new information, instantaneously updating subsequent users with the latest information. Rapid and accurate troubleshooting saves potentially wasted time and eliminates wrong part replacement. Analytical diagnosis and graphical representations ensure that different users of different skills and/or experience levels can efficiently troubleshoot and take needed corrective action. These and other features and advantages will be described in more details herein.

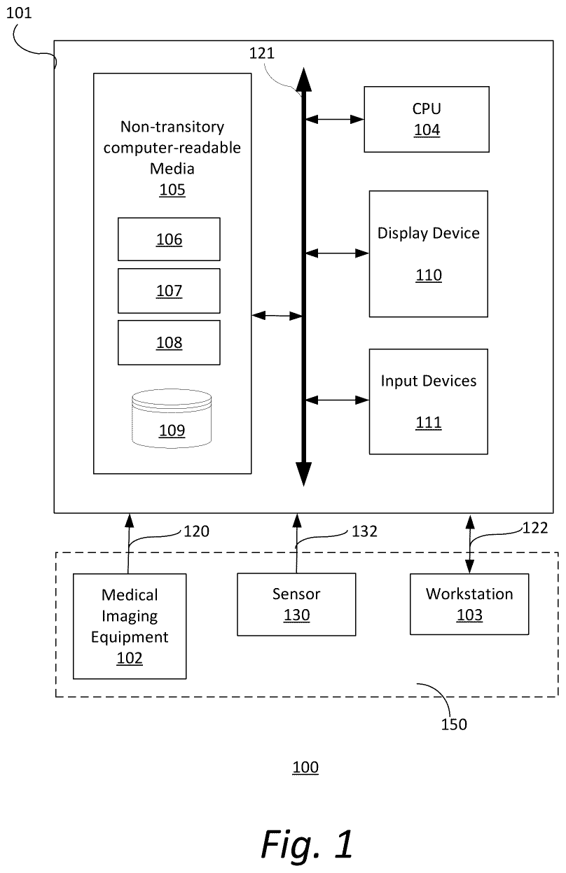

FIG. 1 is a block diagram illustrating an exemplary system 100. The system 100 includes a computer system 101 for implementing the framework as described herein. Computer system 101 may be connected (e.g., using a network) to other machines. In a networked deployment, computer system 101 may operate in the capacity of a server (e.g., thin-client server), a cloud computing platform, a client user machine in server-client user network environment, or as a peer machine in a peer-to-peer (or distributed) network environment. Examples of computer system 101 include, but are not limited to, a personal computer, a laptop, a mobile device, a server or a cloud computing system.

In some implementations, computer system 101 comprises a processor or central processing unit (CPU) 104 coupled to one or more non-transitory computer-readable media 105 (e.g., computer storage or memory), a display device 110 (e.g., monitor) and various input devices 111 (e.g., mouse or keyboard) via an input-output interface 121. Computer system 101 may further include support circuits such as a cache, a power supply, clock circuits and a communications bus (not shown). Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in non-transitory computer-readable media 105. In particular, the present techniques may be implemented by applications including a troubleshooting tool 106, diagnostic tool 107 and pattern creator 108. It should be appreciated that the troubleshooting tool 106, diagnostic tool 107 and pattern creator 108 may be implemented on the same computer system 101 or different computer systems. Additionally, two or more of these applications may be integrated into a single application.

Non-transitory computer-readable media 105 may include random access memory (RAM), read-only memory (ROM), magnetic floppy disk, flash memory, and other types of memories, or a combination thereof. The computer-readable program code is executed by CPU 104 to process data retrieved from, for example, medical imaging equipment 102 and sensor 130. As such, the computer system 101 is a general-purpose computer system that becomes a specific purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein.

The same or different computer-readable media 105 may be used for storing a database (or dataset) 109. Such data may also be stored in external storage or other memories. The external storage may be implemented using a database management system (DBMS) managed by the CPU 104 and residing on a memory, such as a hard disk, RAM, or removable media. The external storage may be implemented on one or more additional computer systems. For example, the external storage may include a data warehouse system residing on a separate computer system, a cloud platform or system, a picture archiving and communication system (PACS), or any other hospital, medical institution, medical office, testing facility, pharmacy or other medical patient record storage system.

Computer system 101 is communicatively coupled to a medical imaging system 150. Medical imaging system 150 includes, for example, medical imaging equipment 102, one or more sensors 130 and a workstation 103. Medical imaging equipment 102 is a radiological imaging modality that acquires medical image data associated with at least one patient. Such medical image data may be processed and stored in database 109. Medical imaging equipment 102 may include a modality that acquires medical image data using techniques such as, but not limited to, high-resolution computed tomography (HRCT), magnetic resonance (MR) imaging, computed tomography (CT), helical CT, X-ray, angiography, positron emission tomography (PET), fluoroscopy, ultrasound, single photon emission computed tomography (SPECT), photoacoustics, microwaves, optical coherence tomography or a combination thereof. Medical imaging equipment 102 may generate log event data 120. Log event data 120 describes occurrences that the medical imaging equipment 102 monitors and records in one or more log files.

One or more sensors 130 acquire state data 132. One or more sensors 130 may be, but is not limited to, a position sensor (e.g., resistance-based, inductive, eddy current-based, capacitive, magnetic, fiber-optic, optical position sensors), pressure sensor, voltage sensor (e.g., resistive, capacitive), temperature sensor (e.g., contact, non-contact), camera (e.g., red-green-blue, infrared, depth, holographic, ultrasound camera), light detection and ranging (LIDAR) sensor, time-of-flight sensor, or a combination thereof. State data 132 may be acquired continuously in a stream (e.g., a stream of temperature measurements). State data 132 may include, but is not limited to, position, voltage, temperature, image, distance, depth, range values or a combination thereof.

Workstation 103 may include a computer and appropriate peripherals, such as a keyboard and display device, and can be operated in conjunction with the entire system 100. For example, the workstation 103 may communicate directly or indirectly with the medical imaging equipment 102 and/or sensor 130 so that the medical image data, log event data 120 and/or state data 132 may be displayed at the workstation 103. The workstation 103 may also provide other types of medical data 122 of a given patient. The workstation 103 may include a graphical user interface to receive user input via an input device (e.g., keyboard, mouse, touch screen, voice or video recognition interface, etc.) to input medical data 122.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

The troubleshooting tool 106, diagnostic tool 107 and pattern creator 108 may be locally installed in a service personnel's computer system (e.g., laptop) or remotely deployed from a cloud platform. Both deployments work in a plurality of modes for troubleshooting: one mode is for instant and live analysis of streaming data (e.g., log events and state data), and another mode for displaying historical data and performing analysis.

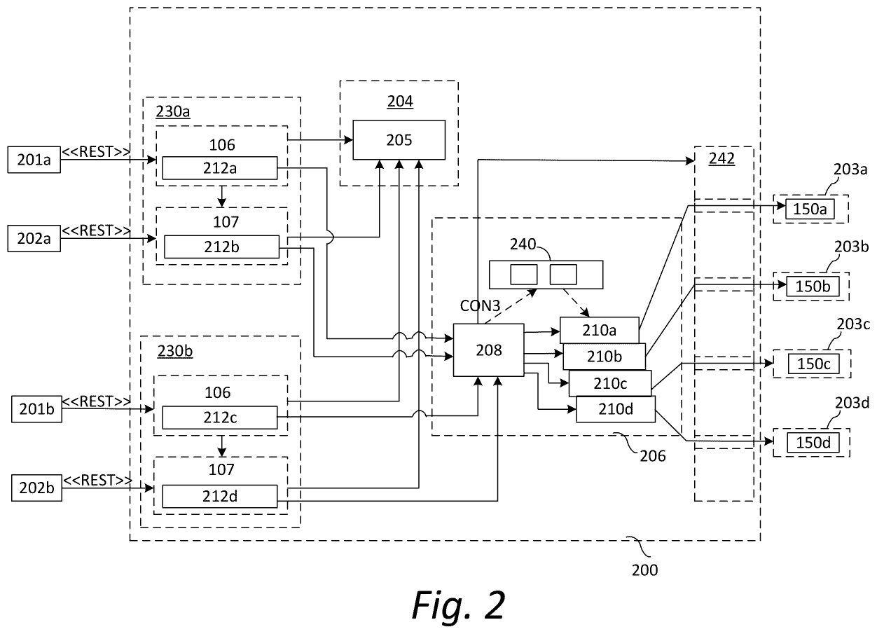

FIG. 2 shows an exemplary cloud platform 200 for remotely deploying the troubleshooting tool 106. Exemplary cloud platform 200 uses WebSocket to provide full-duplex communication channels. Data (e.g., state data) may be communicated via the communication channels using, for example, variable length packets. Each packet may include a single null-terminated string containing a single JavaScript Object Notation (JSON) object (e.g., {“msd”: {“M123456”: {“M112233”: {“M445566”: 26.68}}}).

In some implementations, medical imaging systems 150a-d are coupled to the cloud platform 200 via respective scanner gateways 203a-d. Each medical imaging system 150a-d may also serve as a gateway server. The cloud platform 200 includes, for example, a scanner broker 204 and a scanner proxy 206. Scanner broker 204 includes a broker server 205 for resolving the proxy addresses. Broker server 205 is coupled to the proxy clients 212a-d. Scanner proxy 206 includes a proxy server 208 coupled to gateway clients 210a-d for respective scanner gateways 203a-d. Proxy server 208 is the WebSocket server-side implementation hosted in the scanner proxy 206, while proxy clients 212a-d are the client-side WebSocket connections with the scanner proxy 206. Each gateway client 210a-d is the client side WebSocket connection with each gateway server 150a-d. Due to the statelessness of the WebSocket protocol, mapping 240 is provided to forward gateway response messages from the proxy server 208 to the right gateway clients 210a-d. Mapping 240 may also used to route the client connection to the right tunnel, since two or more sessions to the same system (or same Serial/IVK) can share the same SRS NG tunnel 242. This may be done to avoid creation of multiple tunnels to the same system.

Browsers screens 201a-b and 202a-b may be used to establish cloud connections (or dedicated tunnels) with cloud platform 200 to connect with one of the medical imaging systems 150a-d. Browser screens 201a and 202a may be launched to establish cloud session 230a, and browser screens 201b and 202b may be launched to establish another cloud session 230b. In some implementations, during each cloud session 230a-b, browser screen 201a-b accesses the troubleshooting tool 106, while browser screen 202a-b accesses the diagnostic tool 107 and/or pattern creator 108.

The use of WebSocket enables real-time system representation to a remote service user accessing the troubleshooting tool 106 and the diagnostic tool 107 and/or pattern creator 108 via the browser 202a-d, and facilitates real-time remote communication of state data and log event data from the one or more medical imaging systems 150a-d for real-time troubleshooting and diagnostics. It should be appreciated that other real-time protocols, such as the Transmission Control Protocol (TCP) socket, may also be used.

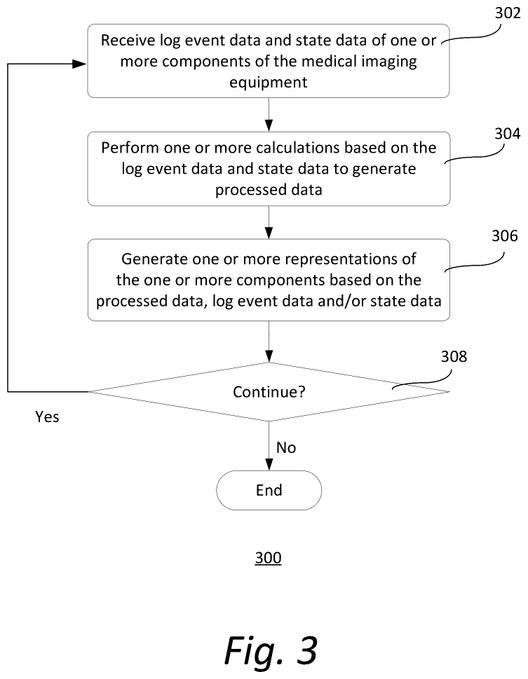

FIG. 3 shows an exemplary method 300 of troubleshooting medical imaging equipment. It should be understood that the steps of the method 300 may be performed in the order shown or a different order. Additional, different, or fewer steps may also be provided. Further, the method 300 may be implemented with the system 100 of FIG. 1, a different system, or a combination thereof.

At 302, log event data 120 and state data 132 of one or more components of the medical imaging equipment 102 are received. The one or more components of such medical imaging equipment 102 may include, but are not limited to, the gantry (e.g., SPECT gantry, CT gantry), uninterruptible power supply (UPS), slip rings, collimators, ECG, cooling system, generator (e.g., X-ray generator), computing device, scanner head, front patient handling system (PHS), rear PHS, electrocardiogram (ECG) scanner, or a combination thereof. Each of these components may also include one or more sub-components.

Log event data 120 and state data 132 may be acquired by medical imaging equipment 102 and sensors 130 respectively. Log event data 120 describes occurrences that the medical imaging equipment 102 monitors and records in a log file. Exemplary log events include, but are not limited to, start and/or end of a task (e.g., diagnostic task, display task), start and/or end of a remote session, error or warning messages (e.g., due to system function or meeting of thresholds), etc.

State data 132 indicates the conditions of the system components and/or sub-components of the medical imaging equipment 102. State data 132 may be continuously acquired at predetermined regular intervals (e.g., 0.5 second interval) to provide the up-to-date information. State data 132 includes, but is not limited to, positional sensor values, voltage values, temperature values, configuration data, or a combination thereof. Configuration data may include information describing the build of the medical imaging equipment 102, such as system options and serial numbers of system component.

The state data 132 may be provided in parallel and synchronized with the log event data 120. The use of a continuous stream of state data 132 and log event data 120 enables real-time analytical calculation, representation and troubleshooting of the medical imaging equipment 102. Additionally, state data 132 and log event data 120 may be stored or cached for performing historical state analysis.

At 304, troubleshooting tool 106 performs one or more calculations based on the log event data 120 and state data 132 to generate processed data. In some implementations, troubleshooting tool 106 performs pattern analysis of the log event data 120 and/or state data 132 in real-time. Such pattern analysis may involve analyzing the logical sequence of events or values (e.g., voltage exceeding a predefined threshold value). Pattern analysis may be applied to the log event data 120 and/or state data 132 to derive milestones or state information. State information describes the current state (e.g., normal, error, warning) of the relevant component. Such pattern analysis may be used to distinguish the root cause from downstream side effects. The advantage of looking at patterns as opposed to individual log event entries or state data values is the ability to identify the root cause.

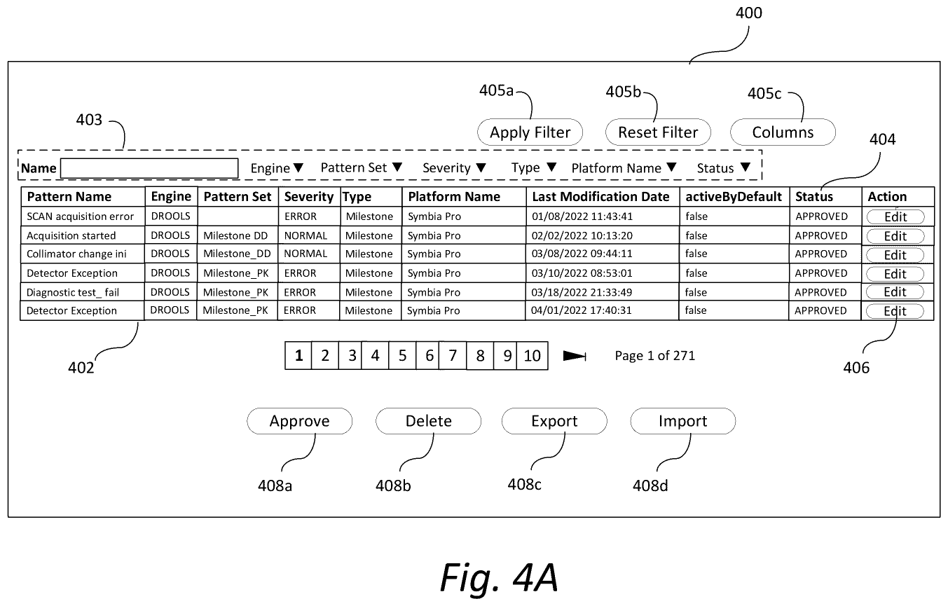

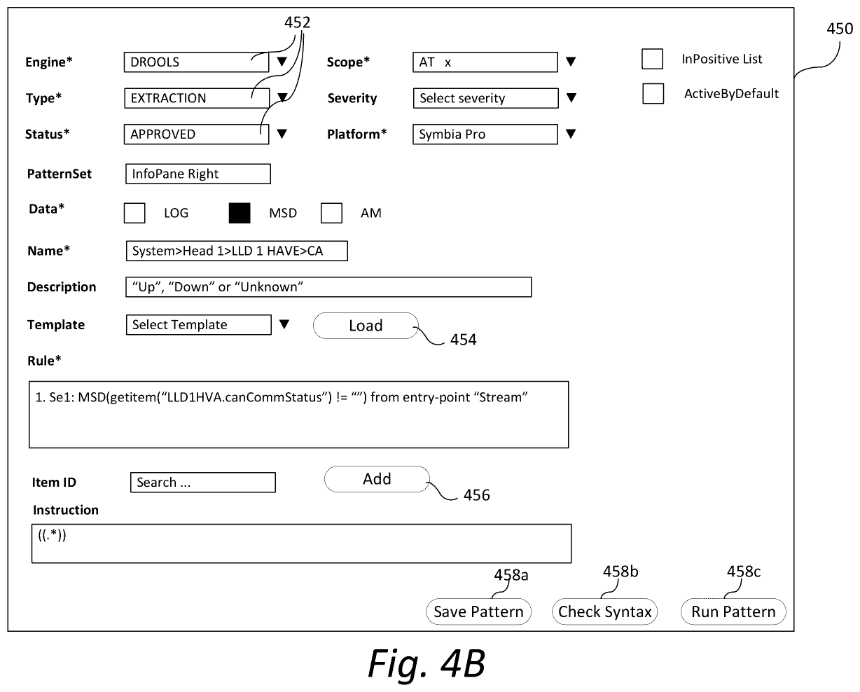

In some implementations, pattern analysis is performed based on pre-defined patterns. Alternatively, machine learning may also be used to perform such pattern analysis. Patterns may be created, edited and/or managed using pattern creator 108. FIG. 4A shows an exemplary user-interface (UI) screen 400 generated by pattern creator 108 to manage patterns. Pattern creator 108 may be conveniently invoked from troubleshooting tool 106 or independently outside troubleshooting tool 106. UI screen 400 presents a table of patterns 402 that have been pre-defined. The pre-defined patterns may reside on a server that can be updated based on new information. The pre-defined patterns may also be used by applications other than the troubleshooting tool 106.

The patterns in the table 402 may be searchable and sortable via interactive UI elements 403 (e.g., text box). Additionally, filters may be applied and reset, and columns may be selected via interactive UI elements 405a-c. Each pattern may be associated with various parameters 404, including but not limited to, name, engine, pattern set, severity, type, platform name, last modification date, active by default, status, and so forth. An interactive UI element 406 (e.g., button) may be provided to enable the user to edit each pattern. Additional interactive UI elements 408a-d may be provided to enable the user to approve, delete, export and import the patterns.

FIG. 4B shows another exemplary user-interface (UI) screen 450 generated by pattern creator 108 to define a pattern. Interactive UI elements 452 (e.g., text boxes, check boxes) are provided to enable the user to define (or select) the values for various parameters of the pattern, such as engine, type, status, pattern set, scope, severity, platform, data, name, description, template, rule, item identifier (ID), instruction, in positive list, active by default, etc. An interactive UI element 454 (e.g., button) may be provided to enable the user to load a template for the pattern. Another interactive UI element 456 (e.g., button) may be provided to enable the user to add an item to the pattern. Additional interactive UI elements 458a-c may be provided to enable the user to save the pattern, perform syntax checking and run the pattern.

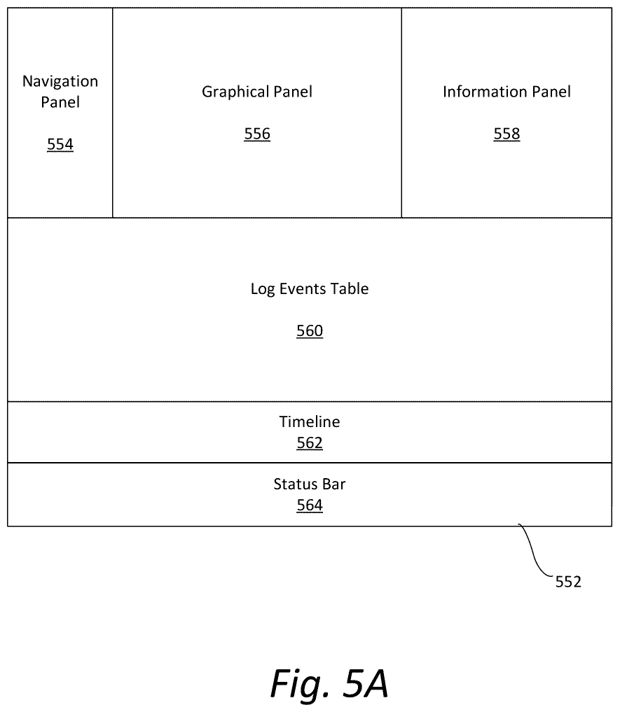

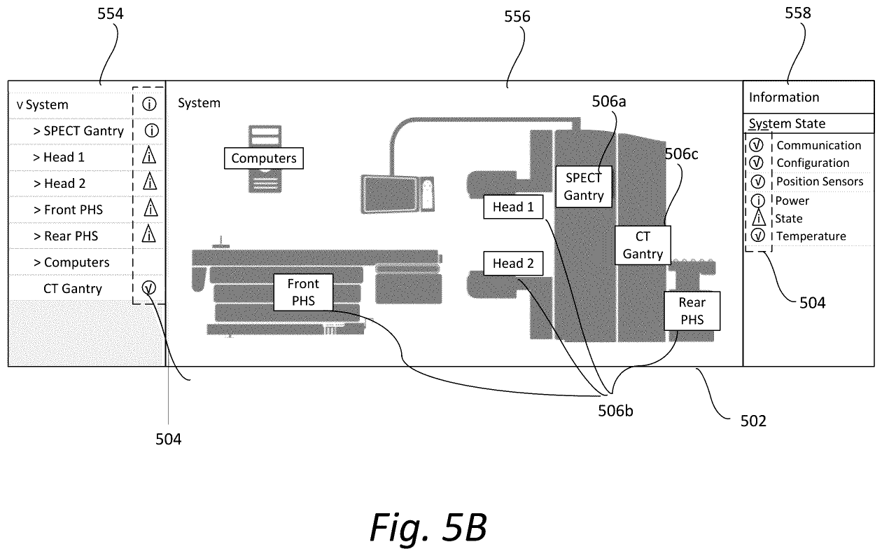

Returning to FIG. 3, at 306, troubleshooting tool 106 generates one or more representations of the one or more components based on the processed data, log event data 120, state data 132 or a combination thereof. FIG. 5A is a block diagram showing an exemplary user interface screen 552 generated by troubleshooting tool 106. In some implementations, the exemplary user interface screen 552 includes a navigation panel 554, a graphical panel 556, an information panel 558, a log events table 560, a timeline 562 and a status bar 564.

In some implementations, the navigation panel 554, the graphical panel 556 and the information panel 558 may be provided in the same row above the log events table 560 to provide a visual overview of the health of the medical imaging equipment 102. Components that need attention (e.g., contains an error or warning) may be highlighted in color (e.g., red or yellow). The log event data 120, state data 132 and corresponding processed information (e.g., state information) may be used to dynamically and continuously update the graphical panel 556, the information panel 558, a log events table 560, a timeline 562 and status bar 564. Status bar 564 presents information associated with the medical imaging equipment 102, including but not limited to, system software version, service license, information of timeline 562 (number of errors, milestones, workflows, search results), etc. When the troubleshooting tool 106 is in “live mode”, the status bar 564 may display an update on what the troubleshooting tool 106 is doing (e.g., receiving data, applying pattern matching).

FIG. 5B shows an exemplary representation 502 generated by troubleshooting tool 106. Exemplary representation 502 includes a navigation panel 554, a graphical panel 556 and an information panel 558. Navigation panel 554 provides a listing of various components and sub-components of the medical imaging equipment 102. State information 504 may be associated with the various components and sub-components in the navigation panel 554. The graphical panel 556 includes graphical representations of the components of the medical imaging equipment 102. Information panel 558 presents the current state information 504 in association with the sensors 130. The state information 504 is derived from the state data 132 and describes the current state (e.g., “error”, “warning”, “normal”).

The state data 132 may be used to dynamically update the navigation panel 554 and the information panel 558. Analytical pattern calculations may be applied to the state data 132 to generate the state information 504. The state information 504 (or states) may be represented by different icons and/or different colors. For example, the state information may be represented using stop light colors (i.e., red=error, yellow=warning, green=normal). Major errors may be highlighted in red to enable the user to immediately focus on the primary components of concern, while warning or causal effects may be identified with yellow as secondary components of concern. In the exemplary representation 502, for instance, there is an error in “power”, and a warning for the “state”, while the “communication”, “configuration”, position sensors” and “temperature” values are all normal.

The state data 132 may also be used to dynamically update the graphical panel 556. In some implementations, colors of labels 506a-c of components in the graphical panel 556 correspond to the state information 504. The colors may include red, yellow and green that correspond to “error”, “warning” and “normal” states of the state information. For example, if the error in “power” is in the SPECT gantry, the label 506a of the SPECT gantry may be depicted in red color. As another example, if the warning for the “state” is in the “head 1”, “head 2”, “front PHS” and “rear PHS”, the corresponding labels 506b may be depicted in yellow color. The other labels 506c of components with no error or warnings may be depicted in green.

The state data 132 may also include configuration data. The navigation panel 554 and the graphical panel 556 may be automatically updated based on the configuration data. Such configuration data may be used to visually depict system configuration, such as graphically identifying (e.g., using different colors) components or sub-components that are installed (or not installed) without the need of extra diagnostics or log events to determine scanner configuration.

Advantageously, the state of the individual components of the medical imaging equipment 102 may be understood from the graphical panel 556 and the information panel 558 without the need to read hundreds of log events. Additionally, state information 504 may also be linked to log events through patterns, so that written confirmation along with action plans for fixing or further troubleshooting may be viewed in, for example, log information.

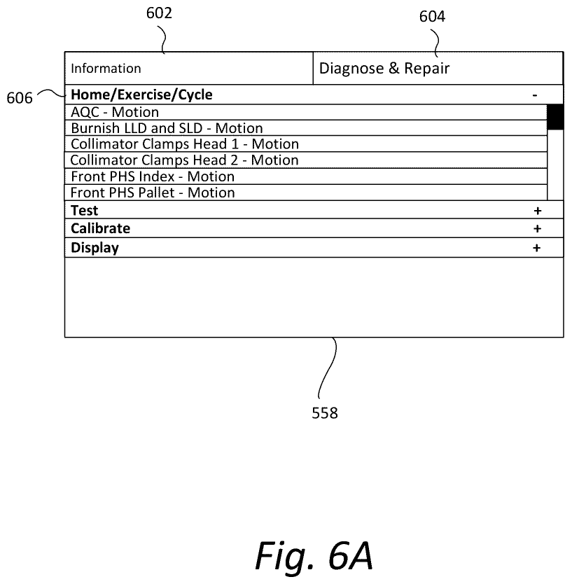

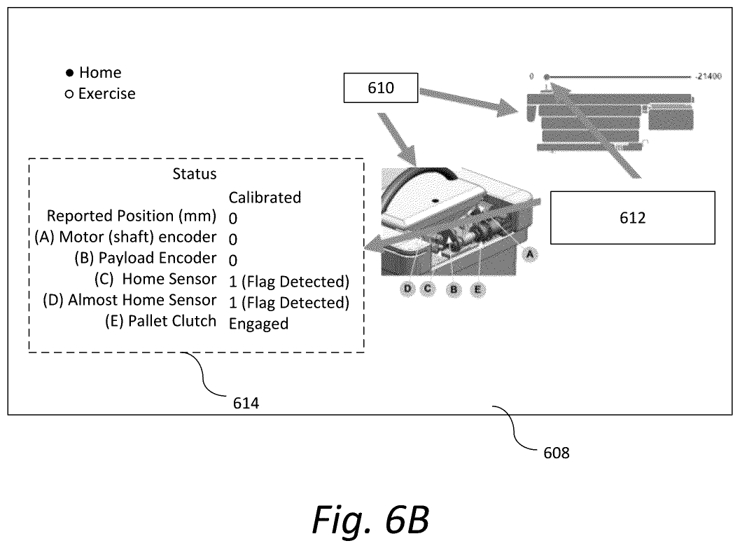

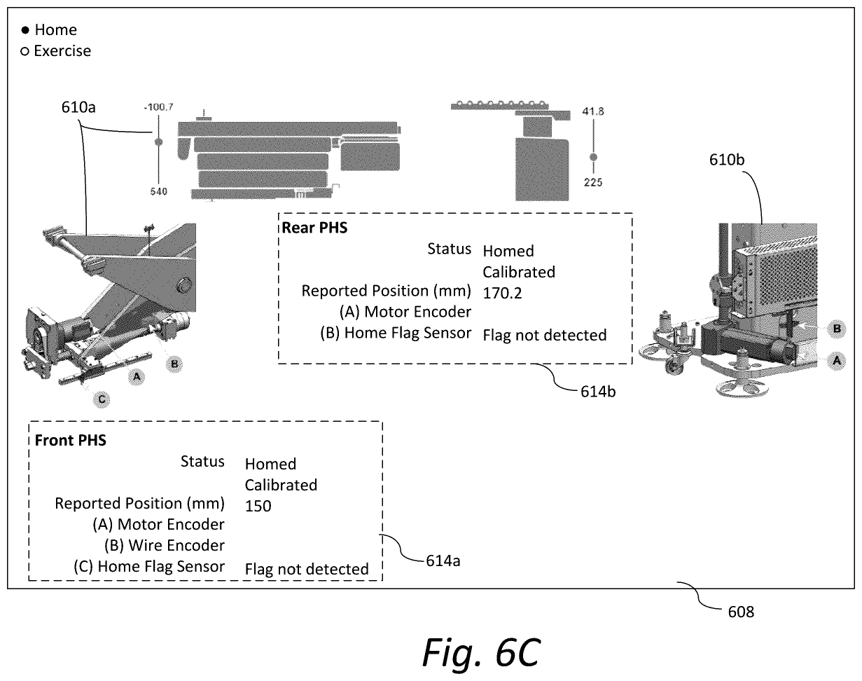

FIG. 6A shows an exemplary information panel 558. Exemplary information panel 558 includes a first tab 602 and a second tab 604. The first tab 602 is selectable to display state information 504 associated with the various components and sub-components of the medical imaging equipment 102, as previously described with respect to FIG. 5B. The second tab 604 is currently selected to launch a diagnostic tool 107 from the troubleshooting tool 106. The diagnostic tool 107 may be launched to trigger diagnostic tasks to be performed by the medical imaging equipment 102 to generate diagnostic results. When the diagnostic tool 107 is launched from the troubleshooting tool 106, the processed data, log event data 120 and state data 132 are still streaming and updating the troubleshooting tool 106. Therefore, while a diagnostic task is triggered by the diagnostic tool 107, the user may switch tabs and still see the user interface screen 552 (e.g., graphical panel 556, information panel 558) being updated in parallel with the task being executed. It should be appreciated that the diagnostic tool 107 may also be launched independently outside troubleshooting tool 106.

Diagnostic tool 107 may be invoked to trigger diagnostic tasks to diagnose and repair components of the medical imaging equipment 102 in response to user input via tab 604. As shown, tab 604 displays various diagnostic tasks 606 that may be performed. Diagnostic tasks 606 include, but are not limited to, home/exercise/cycle, test, calibrate and display. The home/exercise/cycle task causes the components of the medical imaging equipment 102 to move to a predetermined position (e.g., home, exercise or cycle position). The test, calibrate and display tasks cause the medical imaging equipment 102 to perform testing, calibrating and displaying respectively. The diagnostic tool 107 may establish direct tunneling to the medical imaging equipment 102 to execute these diagnostic tasks 606. Advantageously, the diagnostic tool 107 may be used by a user to remotely (i.e., outside the medical imaging system 150) trigger diagnostic tasks to be executed by the medical imaging equipment 102.

FIG. 6B shows an exemplary diagnostic panel 608 generated by diagnostic tool 107. Diagnostic panel 608 includes dynamic graphical representations 610 (e.g., images, computer-generated graphics) of components of medical imaging equipment 102. Dynamic graphical representations 610 are updated to depict the physical locations (e.g., dynamic position relative to axial position/range) of the components. The physical locations may change due to motion from execution of tasks triggered by diagnostic tool 107. For example, in this case, the dynamic graphical representation 610 shows the components in the home position after the “home” function is triggered. Such dynamic graphical representations 610 may be continuously updated using instant feedback of state data 612 acquired from the medical imaging equipment 102. Diagnostic panel 608 may further provide a status box 614 that describes the current status of each of the components (A-E) of interest. Accordingly, diagnostic panel 608 provides instant feedback and facilitates quick understanding of system conditions, enhancing individual diagnostics and enabling corrective actions necessary for troubleshooting. It is particularly useful for facilitating remote troubleshooting.

FIG. 6C shows another exemplary diagnostic panel 608 generated by diagnostic tool 107. Diagnostic panel 608 includes dynamic graphical representations 610a-b. Dynamic graphical representations 610a depict the physical locations of the components of the front PHS and dynamic graphical representations 610b depict the physical locations of the components of the rear PHS. The physical locations may change due to motion from execution of actions triggered by diagnostic tool 107. For example, in this case, the dynamic graphical representations 610a-b show the components in the home position after the “home” function is triggered. Such dynamic graphical representations 610a-b may be continuously updated using instant feedback of state data 612 acquired from the medical imaging equipment 102. Diagnostic panel 608 may further provide status boxes 614a-b that describe the current status of each of the components of interest in the front PHS and the rear PHS.

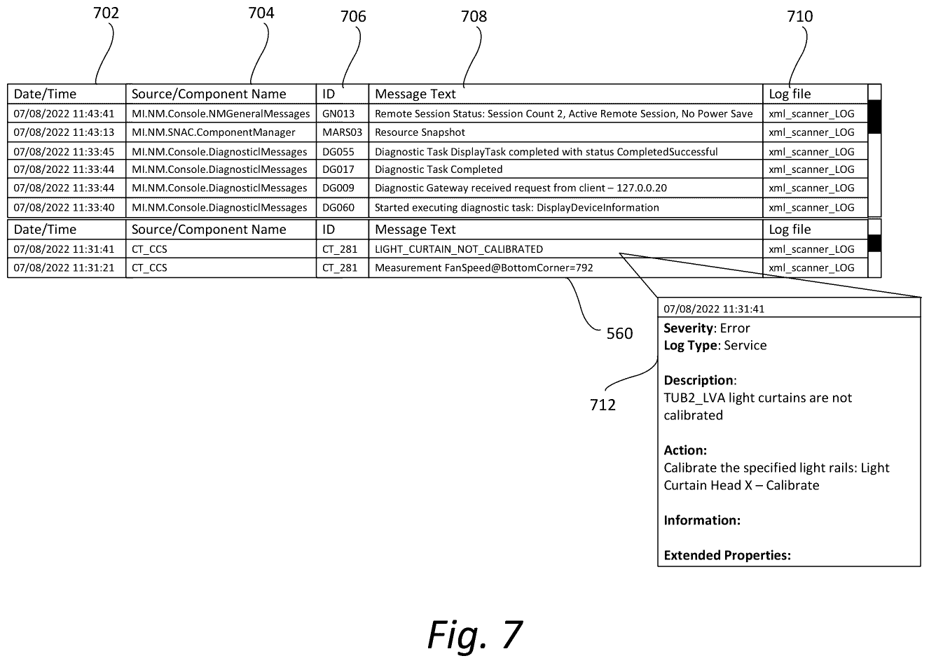

FIG. 7 shows an exemplary log events table 560. Log events table 560 presents information about each log event, including, but not limited to, date and time 702, source (or system component) name 704, identifier (ID) 706, message text 708, log file 710, or a combination thereof. Each log event may be expanded to display a text box 712 that presents additional information, such as a detailed description of the event and a recommended action. Log events of interest in the log events table 560 may be highlighted. In some implementations, log events are highlighted in different colors based on severity (e.g., “error” is red, “warning” is blue, “for information” is black, “success” is green). Log events of interest may be identified based on pre-defined patterns.

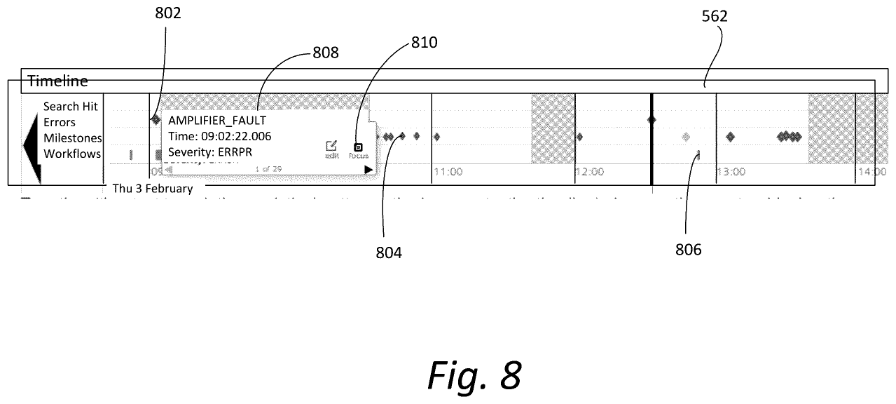

FIG. 8 shows an exemplary timeline 562. Timeline 562 provides a high level view of the history of log events depicting usage of the medical imaging equipment 102. Exemplary timeline 562 provides a quick snapshot view of historical log events of interest identified through pattern analysis. Such log events include, for instance, errors, milestones or workflow events along a continuous timeline. For example, if the log event matches a pre-defined pattern, a “milestone” symbol 804 (e.g., green diamond) or an “error” symbol 802 (e.g., red diamond) may be added to the timeline 562. If two sequential log events match two pre-defined patterns, a bar 806 in the timeline may be generated to depict workflow events (e.g., acquisition). Primary log events of interest may be highlighted on the timeline 562 as well as the log events table 560. The representations in the graphical panel 556 and the timeline 562 may be synchronized to enable the user to focus on log events of interest.

Additionally, a text box 808 may be displayed to describe the event (e.g., name, time stamp, severity). In some implementations, text box 808 presents an interactive user interface element 810 to enable the user to focus on (or interrogate) the particular event for a deep understanding of issues and any actions needed to correct the error. Focusing on a particular event updates the graphical panel 556 and information panel 558 with state data and associated state information that matches the timestamp of the event. Since state data is periodically updated (e.g., every 0.5 second), focusing on a milestone updates the graphical panel 556 and information panel 558 to reflect the system state within a predetermined period (e.g., 0.5 seconds) of the log event timestamp.

Returning to FIG. 3, at 308, it is determined if the method 300 continues. The method may be determined to continue if, for example, the service personnel is still performing troubleshooting of the medical imaging equipment 102. If yes, steps 302 through 306 are repeated to continue supporting troubleshooting of the medical imaging equipment 102. If no, the method 300 ends.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. One or more non-transitory computer readable media embodying instructions executable by machine to perform operations for troubleshooting a medical imaging equipment, the operations comprising:
triggering one or more diagnostic tasks to be performed by one or more components of the medical imaging equipment;
acquiring, by one or more sensors, state data of the one or more components of the medical imaging equipment;
performing one or more calculations based on the state data to generate processed data; and
generating one or more representations of the one or more components of the medical imaging equipment based on the processed data, the state data, or a combination thereof.

2. The one or more non-transitory computer readable media of claim 1 wherein the performing the one or more calculations based on the state data comprises performing pattern analysis to generate state information.

3. The one or more non-transitory computer readable media of claim 2 wherein the performing the pattern analysis comprises performing analysis based on one or more pre-defined patterns.

4. The one or more non-transitory computer readable media of claim 1 wherein the one or more representations comprise a graphical panel that includes one or more graphical representations of the one or more components that are dynamically updated based on the state data.

5. The one or more non-transitory computer readable media of claim 1 wherein the one or more diagnostic tasks comprise moving to a predetermined position, testing, calibrating, displaying, or a combination thereof.

6. A troubleshooting system, comprising:
one or more sensors that acquire state data of one or more components of a medical imaging equipment; and
a computer system in communication with the one or more sensors, wherein the computer system includes
a non-transitory memory device for storing computer readable program code, and
a processor in communication with the memory device, the processor being operative with the computer readable program code to perform operations including
performing one or more calculations based on the state data and log event data to generate processed data; and
generating one or more representations of the one or more components of the medical imaging equipment based on the processed data, the log event data, the state data, or a combination thereof.

7. The troubleshooting system of claim 6 wherein the one or more sensors comprise a position sensor, a voltage sensor, a temperature sensor, a camera, a light detection and ranging (LIDAR) sensor, a time-of-flight sensor, or a combination thereof.

8. The troubleshooting system of claim 6 wherein the medical imaging equipment comprises a modality that acquires medical image data using high-resolution computed tomography (HRCT), magnetic resonance (MR) imaging, computed tomography (CT), helical CT, X-ray, angiography, positron emission tomography (PET), fluoroscopy, ultrasound, single photon emission computed tomography (SPECT), photoacoustics, microwaves, optical coherence tomography, or a combination thereof.

9. The troubleshooting system of claim 6 wherein the one or more components comprise a gantry, a slip ring, a cooling system, a generator, a computing device, a scanner head, a front patient handling system (PHS), a rear PHS, or a combination thereof.

10. The troubleshooting system of claim 6 wherein the state data comprises positional sensor values, voltage values, temperature values, configuration data, or a combination thereof.

11. The troubleshooting system of claim 6 wherein the one or more sensors continuously acquire the state data at predetermined regular intervals.

12. The troubleshooting system of claim 6 wherein the processor is operative with the computer readable program code to performing the one or more calculations based on the state data comprises performing pattern analysis to generate state information.

13. The troubleshooting system of claim 6 wherein the one or more representations comprise a user interface with an information panel and a graphical panel that includes one or more graphical representations of the one or more components, wherein the information panel and the graphical panel are dynamically updated by the state data.

14. The troubleshooting system of claim 13 wherein the user interface further comprises a navigation panel that is dynamically updated by the state data.

15. The troubleshooting system of claim 13 wherein the user interface further comprises a log events table that highlights log events of interest.

16. The troubleshooting system of claim 13 wherein the user interface further comprises an interactive timeline that highlights log events of interest.

17. The troubleshooting system of claim 6 the processor is further operative with the computer readable program code to trigger one or more diagnostic tasks to be performed by the medical imaging equipment.

18. The troubleshooting system of claim 17 the one or more diagnostic tasks comprise moving the medical imaging equipment to home, exercise or cycle positions.

19. The troubleshooting system of claim 6 wherein the state data is communicated between the one or more sensors and the computer system using WebSocket.

20. A method for troubleshooting a medical imaging equipment, comprising:
receiving log event data, state data of one or more components of the medical imaging equipment;
performing one or more calculations based on the log event data and the state data to generate processed data; and
generating one or more representations of the one or more components of the medical imaging equipment based on the processed data, the log event data, the state data, or a combination thereof.
